(19) United States
(12) Patent Application Publication
(10) Pub. No.: US 2016/0022733 A1
(43) Pub. Date: Jan. 28, 2016

(54) NOVEL FORMULA OF IRON BASED NANOCOMPOSITES FOR RAPID AND EFFICIENT TREATMENT OF IRON DEFICIENCY ANEMIA

(71) Applicants: Mona Bakr Mohamed MAHMOUD, (US); Sherine Hassan Abbas HELMY, (US); NANOTECH FOR PHOTOELECTRONICS RESEARCH, Giza (EG); EUROPEAN EGYPTIAN PHARMACEUTICAL INDUSTRIES (EEPI), Alexandria (EG)

(72) Inventors: Mona Bakr Mohamed MAHMOUD, Alexandria (EG); Sherine Hassan Abbas HELMY, Alexandria (EG)

(21) Appl. No.: 14/772,648

(22) PCT Filed: Oct. 29, 2013
(86) PCT No.: PCT/EG2013/000027

§ 371 Date: Sep. 3, 2015

(30) Foreign Application Priority Data
Mar. 6, 2013 (EG) 2013030371

(51) Int. Cl. A61K 33/26; A61K 9/48; A61K 31/375; A61K 9/00; A61K 31/519; A61K 31/455

(52) U.S. Cl. CPC A61K 33/26; A61K 31/519; A61K 31/455; A61K 31/375; A61K 9/0053; A61K 9/48

(57) ABSTRACT

New formulas of Iron oxides nanoparticles capped with a mixture of multivitamins such as folic acid, Nicotinic acid (vitamin B9) and Ascorbic acid (vitamin C) has been developed for the rapid and efficient treatment of life threatening iron-deficiency anemia. Small single dose of iron oxides-multivitamin nano-composite as low as 8.3 mg elemental iron per does is sufficient to increase the hemoglobin level from 4.4 g/dl up to 14.6 g/dl within only four days after administration. The multivitamin which used in this nano-composite enhances iron absorption significantly and elevated the concentration of hemoglobin. Two dosage forms of Iron nano-composites have been developed, gel capsules and aqueous solution for oral administration. Animal trials studies reveal that introducing single dose of Iron Oxide-vitamin nano-composites containing 8.6 mg elemental iron per kg rat body weight is sufficient to correct the hemoglobin level and cure Anemia via oral administration.

### TECHNICAL FIELD

Iron is a component of proteins required for crucial cellular processes. Iron-containing proteins have essential roles in oxygen transport, ATP production, DNA synthesis and other physiological processes [1-3]. Low consumption of foods rich in bioavailable iron (Fe) such as red meat or hemorrhage are the main causes of iron deficiency Anemia. The symptoms of iron deficiency anemia include dyspnea, headaches, light-headedness, short breath, fatigue, be forgetful, feel grouchy, lose appetite and weight and have trouble concentrating. All of these symptoms appear as a result of the reduction in the oxygen-carrying capacity of red blood cells. Therefore, anemia has been associated with reduced health-related quality of life and its treatment is essential to improve our health and performance.

It is estimated that 50% of pregnant women in developing countries have iron deficiency anemia (IDA), while reports from the World Health Organization (WHO) estimate that 46% of the world's 5- to 14-year-old children, majority of them are from the developing world. In Egypt, anemia remains a problem that suggests the need to expand the iron strategies for the whole country and not only addressing the iron needs of Egyptian children and pregnant women.

### BACKGROUND ART

Although iron is extremely important, the iron overload and significant increase in iron stores can be toxic to the body. Scientists revealed that iron can increase lipid peroxidation and lead to cancer formation. The process of lipid peroxidation is initiated by reactive oxygen species, such as hydroxyl radicals, and stimulated by excess iron ions. On the other hand, iron-deficient rats rapidly accumulate copper eightfold higher than those in normal rats, and consequently, excess copper can also catalyze lipid peroxidation as iron.

Additionally, copper- or iron-deficient rats have been shown to accumulate triglycerides in liver and plasma. High concentrations of triglycerides provide more lipid substrate for lipid peroxidation, and this may have contributed to the high levels of liver and kidney malondialdehyde in deficient rats.

Furthermore, several mechanisms have been proposed by which iron overload may influence the development of cancer. It has been suggested that excess iron may alter immune status and act as a co-carcinogen as well a catalyst for hydroxyl radical production and lipid peroxidation. Reactive oxygen species are considered to be carcinogenic because of their ability to produce DNA adducts, DNA strand breaks, and to modulate gene expression. High dietary iron levels could also enhance oxidative stress by interfering with absorption of other minerals as copper and manganese (Mn), in which their depletion may result in decreased antioxidant enzyme activity, particularly that of copper-zinc (Cu, Zn) and manganese superoxide dismutase (SOD). Taking together, to address the problem of iron-deficiency anemia, iron supplements should be prescribed especially for children and pregnant women, but under recommendation. Because iron is difficultly excreted outside the body, a lot of care should be taken in order to avoid cellular iron accumulation and generation of lipid peroxidation, oxidative stress and cancer. These challenges raise the need for a new form/preparation that can 1)—secure the iron availability in the body through its high absorption rate 2)—has the ability to treat the iron-deficiency anemia in short time-course 3)—has the high efficacy to overcome the life-threatening situation due to significantly low-Hb levels.

To meet these challenges, we made new formulation of nano-sized iron oxide particles with three different vitamins, Vitamin B9 (folic acid) and vitamin B3 (Nicotinic acid) are essentials for all cell formation, and Vitamin C (ascorbic acid) is antioxidant, enhances iron absorption and protects against the clastogenic effect of iron [4]. It is known that the biochemical function of nicotinic acid is its synthesis to the pyridine nucleotides and subsequent role in the cell respiration. Immature nucleated erythrocytes respire and presumably, must utilize the pyridine nucleotides in this respiration. Since, the lifetime of the erythrocyte appears to be very short, the requirement of nicotinic acid for the demands of erythropoiesis is great. Thus, as the supplies of Nicotinic acid diminish, anemia might develop due to the lack of cozymase in the earliest stage of cell development [5].

When body iron stores are depleted (low-serum iron, serum ferritin and transferrin saturation), erythropoiesis becomes iron restricted. This can be reflected by a low mean corpuscular volume and mean corpuscular haemoglobin, an increase in the percentage of hypochromic red cells and low haemoglobin (Hb) content of reticulocytes [6]. Administration of oral iron can correct the Hb, provided time is not a limitation, and significant doses (200 mg) can be tolerated. Following the administration of oral iron, it takes 2-3 weeks for the Hb to start rising, 2 months for it to return to normal levels and 6 months for iron stores to be repleted; with intravenous iron Hb starts rising in 1 week, the percentage of responding patients is higher and iron stores are repleted [7]. Boosting iron stores is an advantage, particularly for patients receiving Erythropoiesis Stimulating Agents (ESAs) [8].

There are now three iron products that are safe for intravenous administration: iron gluconate, iron saccharate, and low molecular weight iron dextran. The best gives rise in the HP level after one week, and it comes back to the normal level after a month of treatment through twice weekly intravenous administration does.

We discovered that iron oxides nano-composites capped with Folic acid (vitamin B9), Nicotinic acid (vitamin B3), and/or Ascorbic acid (vitamin C) can be used as new formula or food supplementary for Anemia treatment and it rise the RBCs and correct the HP level to their normal values within less than a week.

The aim of this study is to test the ability of single doses Nano-sized iron oxide (Magnetite) particles capped with vitamin mixture (folic, nicotinic and ascorbic acids) to treat life-threatening iron deficiency anemic rats within one week. The results were compared with Ferric Chloride treated group (The parent iron source) in order to measure and differentiate between the efficacies of Nano- and Micro-sized iron supplements. We prepared iron oxides nanoparticles capped with different vitamins. We discovered that these nano-composites increases the Hemoglobin level and also increase the formation of RBCs.

Animal trial experiment has been carried out to ensure the efficiency of iron oxide nanoparticles capped with Vitamin B3, B9 and C in iron deficiency Anemia treatment. The hemoglobin level has been decreased in the mammals to be 4.6 g/dl, and then single dose of our proposed new formulation of the drug was introduced to the mammals orally. Within 4 days, the Hemoglobin level and RBCs comes back into normal.

### DISCLOSURE OF INVENTION

Part I: Preparation of Nano-sized Iron Nano-Composite:
Preparation of biocompatible nano-sized iron oxides capped with a mixture of vitamins (Folic acid, Nicotinic acid and ascorbic acid).
Determination of the particle size and shape using Transmission Electron Microscope (TEM) images. TEM images as shown in FIG. 1.
Quantitative determinations of the iron content of the prepared stock solution of the elemental iron in the nano-composite using Inductivity Coupled Plasma (ICP).

Part II: Animal Trial Experimental Design
Five weeks old Albino rats of the Wisconsin Holtzman strain, weighing 150-160 g, were divided into six, as showing below

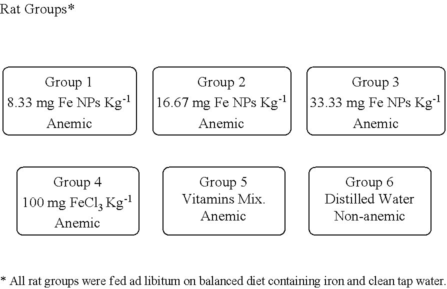

All methods used in this study were done according to the NIH guidelines.

1. Induction of Hemorrhagic Anemia

Rats of groups 1-5 were exposed daily to blood withdrawal from inner canthus of eyes. The hemoglobin concentration of collected blood samples in heparinized tubes was measured by colorimetric method using commercially available kit (Drabkin's solution). The blood withdrawal was stopped when rat lives were threatened and hemoglobin reached the range of 4 -5 g/dL in all lab animals. Rats of control group were not exposed to any blood withdrawal.

2. Doses and Treatments

After induction of Life-threatening hemorrhagic anemia, all rats were fasted for 6 hours prior their oral administration with different treatments and doses. Groups 1-3 were administered 1.0 ml/rat of Nano-sized iron (Magnetite) of different concentrations (0.86, 1.7, and 3.4 mg Kg⁻¹ respectively rat body weight, which equivalent to 8, 16, 33 mg dose in human, respectively), while group 4 was administered 10.2 mg Kg⁻¹ of Ferric chloride (1 ml/Rat; the parent material of magnetite). Two control groups were allocated for this pilot experiment; group 5 where rats were exposed to Life-threatening hemorrhagic anemia and treated with the vitamins mixture dissolved with Nano sized magnetite (Ascorbic, folic and nicotinic acids), and group 6 where rats were non-anemic and administered distilled water (Table 1).

TABLE 1
Treatments, doses and hemoglobin concentration of
rat groups (n = 3 Rats)

| Groups* | Treatment and Dose |
|---|---|
| Group 1 | [symbol] |
| Group 2 | Magnetite 16.67 mg/Kg Rat weight |
| Group 3 | [symbol] |
| Group 4 | Ferric Chloride 100 mg/Kg Rat weight |
| Group 5 | [symbol] |
| Group 6 | Distilled Water |

[symbol]

indicates data missing or illegible when filed

3. Hemoglobin Concentration After Different Treatments

Rats of different groups were left for 3 days after oral administration of different treatments, fed ad libitum on balanced diet containing iron and clean tap water. Blood samples were collected on 4^{th} and 7^{th} day after dosing and Hb concentration was measured using colorimetric method.

Experimental Results

TABLE 2
Hemoglobin concentration of rat groups during induction
of Life-threatening hemorrhagic anemia and at start of
all treatments

| | Hb conc. during | | | | Hb. Conc. |
|---|---|---|---|---|---|
| | anemia induction | | | | @ start |
| Groups* | 31/10 | 4/11 | 5/11 | 6/11 | 8/11/2012 |
| Group 1, Magnetite 8.33 | 11.81 | 9.9 | 7.4 | 6.4 | 4.4 g/dL |
| Group 2, [symbol] | [symbol] | [symbol] | [symbol] | [symbol] | [symbol] |
| Group 3, Magnetite 33.33 | 11.72 | 9.9 | 7.8 | 5.8 | 4.3 g/dL |
| Group 4, [symbol] | [symbol] | [symbol] | [symbol] | [symbol] | [symbol] |
| Group 5, Vitamins mix | 11.53 | 9.4 | 6.8 | 5.8 | 4.4 g/dL |
| Group 6, [symbol] | [symbol] | [symbol] | [symbol] | [symbol] | [symbol] |

[symbol]

indicates data missing or illegible when filed

TABLE 3
Hemoglobin concentration of rat groups at 1 day
before, 4 and 7 days after all treatments

| | Hb conc. (g/dL) before and after Treatments | | |
|---|---|---|---|
| Groups* | 1 day Before | 4 Days After | 7 Days After |
| Group 1, Magnetite 8.33 | 4.4 | 12.2 | 14.6 |
| Group 2, [symbol] | [symbol] | [symbol] | [symbol] |
| Group 3, Magnetite 33.33 | 4.3 | 11.2 | 14.1 |
| Group 4, [symbol] | [symbol] | [symbol] | [symbol] |
| Group 5, Vitamins mix | 4.4 | 9.6 | 12.7 |
| Group 6, [symbol] | [symbol] | [symbol] | [symbol] |

[symbol]

indicates data missing or illegible when filed

Results Could be Summarized as Follow:
A. As described in Table 3, treatment of Life-threatening iron-deficiency anemic rats with different doses of Nano-sized iron supplements resulted in significant elevation of hemoglobin concentration within just 4 days after magnetite administration.
B. The hemoglobin concentration results in magnetite-treated groups exceeded the figures of all other groups including the control non-anemic one after 7 days post treatment.
C. The dose of 0.8 mg magnetite was able to correct the concentration of hemoglobin in anemic rats better than the two other doses of 1.7 and 3.4 mg after the 4^{th} days post administration. The HB level raise from 4.4 up to 14.6 g/dl within seven days and stabilized at 13.6 for more than 80 days after administration.
D. Administration of Ferric Chloride to anemic rats corrected the Hb. concentration but achieved neither the levels in Nano-sized magnetite groups nor the level in control non-anemic rats.
E. Administration of vitamins mixture (Ascorbic, Folic and Nicotinic acids) to anemic rats was able to increase the absorption of iron significantly and elevated the concentration of hemoglobin but it didn't reach or exceed the same levels of any other group.
F. The results of histopathological examination of Liver, spleen, duodenum, kidneys and brain did not show any sign of toxicity.
G. Depletion was observed in the RBCs precursor after induction of Anemia, which has been corrected after single dose nano-sized iron composite as shown in FIG. 4.

### CONCLUSION

The single dose of 0.83 mg/kg rat body which equal to 8.3 mg in human of nano-sized iron oxide nanoparticles capped with a mixture of vitamins (B3, B9 and C) regained the anemia due to iron deficiency in less than 4 days. The used dose is apparently safe as it is 1660 times less than the LD50 of Magnitite nanoparticles in human. As well magnetite nanoparticles has been already FDA approved (10)

### REFERENCES

1. J. L. Beard, H. Dawson, D. Pinero, Iron metabolism: a comprehensive review, Nutr. Rev. 54 (1996) 295-317.
2. E. R. Monsen, Iron nutrition and absorption: dietary factors which impact iron bioavailability, J. Am. Diet. Assoc. 88 (1988) 786-790.
3. R. J. Wood, O. J. Han, Recently identified molecular aspects of intestinal iron absorption, Nutrition 128 (1998) 1841-1844.
4. ACC/SCN second report on the world nutrition situation 1992, vol. 1. Global and regional results. Geneva: ACC/SCN WHO, 1992. , H. Tapiero, L. Gate, K. D. Tew, Iron: deficiencies and requirements, Biomed. Pharmacother. 55 (2001) 3
5. J. D. Cook, Diagnosis and management of iron-deficiency anaemia, Best. Pract. Res. Ha. 18 (2005) 19-332
6. A. Maniatis, The role of iron in anaemia management: can intravenous iron contribute to blood conservation? ISBT Science Series (2008), 3(1), 139-143.
7. Fantini, Ana Paula; Canniatti-Brazaca, Solange Guidolin; Souza, Miriam Coelho; Mansi, Debora Niero Ciencia e Tecnologia de Alimentos (Campinas, Brazil) (2008), 28(2), 435-439.
8. J. M. Kim, C H Ihm, H. J. Kim: Evaluation of reticulocyte haemoglobin content as marker of iron deficiency and predictor of response to intravenous iron in haemodialysis patients. Int. J. Lab. Hematol. 2008; 30:46-52
9. R Agarwal, A R Rizkala, B Bastani M O, Kaskas, D J Leehey, A Besarab: A randomized controlled trial of oral versus intravenous iron in chronic kidney disease. Amer. J. Nephrol. 2006; 26 :445-454, C. Brugnara, L A Chambers, E Malynn, M A Goldberg, M S Kruskall: Red blood cell regeneration induced by subcutaneous recombinant erythropoietin: iron deficient erythropoiesis in iron replete subjects. Blood, 1993; 81:956-964
10. L. X. Tiefenaure, in T. Vo-Dinh (Ed. 2007), Nanotechnology in Biology and Medicine: Methods, Devices, and Application, Vol. Section D: Nanomedcine Applications D1, CRC Press, Taylor and Francis, Boca Raton, Fla., USA, P1.

### BRIEF DESCRIPTION OF THE DRAWINGS

FIG. 1: Demonstration of the Patent Idea (novel formula of iron based nanocomposites for rapid and efficient treatment of iron deficiency anemia).

FIG. 2: TEM images of nano-sized iron oxide nanoparticles capped with a vitamins mixture (B3, B9 & C).

FIG. 3: graphical illustration of anemia induction in all groups except the control

FIG. 4: BB level after anemia induction, then single dose treatment with 8.3 mg iron nano-composite with vitamins.

FIG. 5: Bone-marrow section of the anemic rat (left) and after introducing single dose of nano-sized iron-Vitamins composite (right), Vitamin mixtures (B3, B9 and C) used as stabilizer and capping for iron nanoparticles.

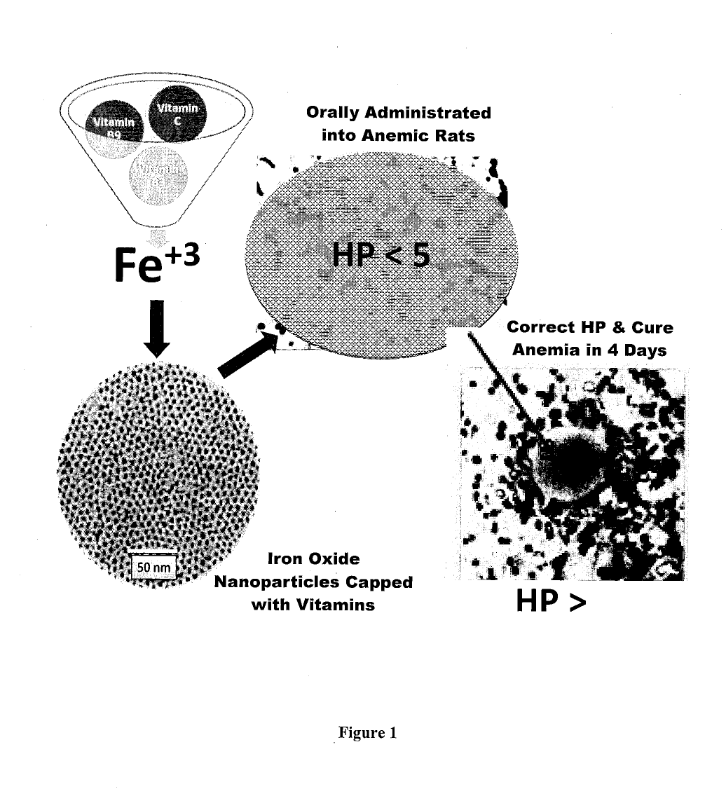

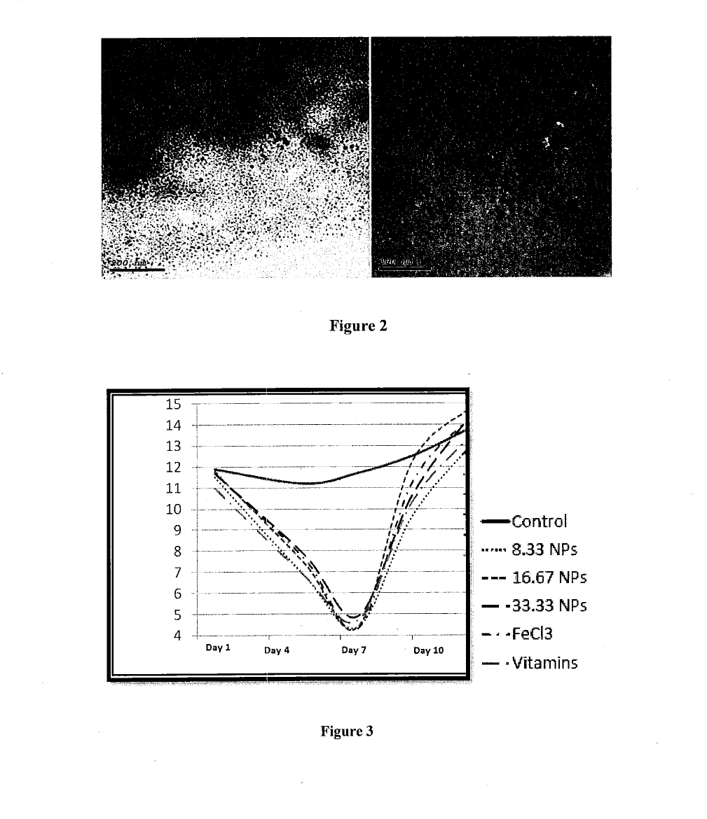

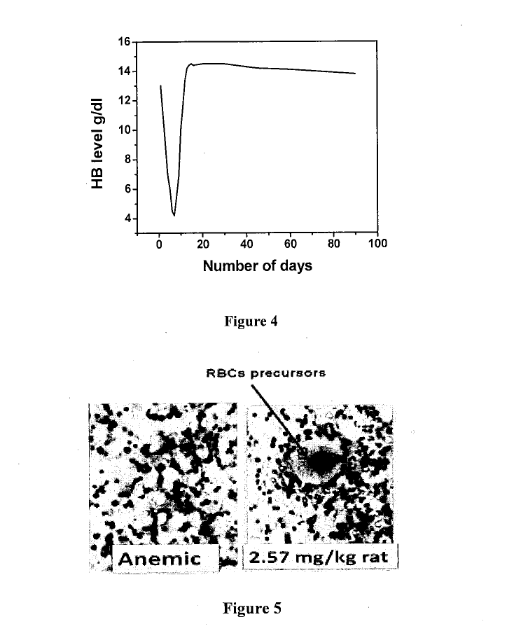

1- A novel formulation for Anemia treatment based on Biocompatible iron oxides nano-composites capped with Folic acid, Nicotinic acid and ascorbic acid

2- Nano-sized iron Composite prepared in (1) could be administrated orally.

3- Two dosage forms of the drug have been formulated, each containing 25 mg elemental iron, gel capsules and aqueous solution and both formulation for oral administration.

4- Single dose of our new formula which contains nano-sized iron oxides especially Fe₂O₃ nano-composites capped with a mixture of folic acid, nicotinic acid and ascorbic is sufficient for Anemia treatment. It enhance the hemoglobin (Hb) level from 4.4 g/dL to 12.2 g/dL within 4 days after administration, then raise to 14.6 g/dL after seven days. The Hb level stabilizes for more than three months (along all the period of the experiment).

5- The nano-sized iron based nano-composite capped with multivitamin stimulates the bone-marrow and the production of RBCS without any apparent toxicity.

6- The effective dose is 1660 times less than the LD50, and two times less than the daily dose of Iron in any food supplementary.
